(19) United States
(12) Patent Application Publication
(10) Pub. No.: US 2014/0357841 A1
(43) Pub. Date: Dec. 4, 2014

(54) Method for stabilizing polypeptide into alpha helix

(71) Applicant: Peking University Shenzhen Graduate School, Shenzhen (CN)

(72) Inventors: Zigang Li, Shenzhen (CN); Qingzhou Zhang, Shenzhen (CN)

(73) Assignee: Peking University Shenzhen Graduate School, Shenzhen (CN)

(21) Appl. No.: 14/164,246

(22) Filed: Jan. 26, 2014

(30) Foreign Application Priority Data
May 29, 2013 (CN) 201310205762.0

(51) Int. Cl. C07K 1/113

(52) U.S. Cl. CPC C07K 1/1136
USPC 530/333; 530/300

(57) ABSTRACT

A method for stabilizing an alpha helix of a polypeptide includes steps of: (1) connecting an unnatural amino acid to an amino terminus of the polypeptide and end-capping via an acetylation; (2) processing a product of the step (1) with a thiolene reaction and obtaining a polypeptide compound having a modification of thioether side chains; (3) oxidizing the polypeptide compound having the modification of the thioether side chains, and obtaining a polypeptide compound having a modification of R-configured sulfoxide side chains or S-configured sulfoxide side chains; (4) separating and purifying a product of the step (3), and obtaining the modification of the R-configured sulfoxide side chains. CD diagrams show that, via chiral sulfoxide side chains, the method has good performance on stabilizing the alpha helix of the polypeptide and good tolerance to a polypeptide sequence.

### BACKGROUND OF THE PRESENT INVENTION

1. Field of Invention

The present invention relates to a field of polypeptide structure stabilization technology, and more particularly to a method for stabilizing a polypeptide into an alpha helix by constructing side chains and a polypeptide compound having a modification of the side chains obtained by the method.

2. Description of Related Arts

According to the prior researches, only a small sequence in the protein contributes to binding, while the rest parts contribute to the structural specificity of the sequence. As one of the most important secondary conformations, the alpha-helix conformation plays a key role in the cellular physiologic process. For example, the protein-protein mutually functioned zones are mostly the alpha-helix conformations. Besides, stabilizing the alpha-helix conformation of the polypeptide greatly improves the protein-degradation resistance and membrane penetration ability of the polypeptide.

The researchers have developed various polypeptide conformation stabilization technologies for making the polypeptides have stable alpha-helix conformation, such as the salt bridge, the metal-chelating, HBS, and the construction of covalent side chains. The construction of the covalent side chains is relatively common, and the formation of the amido bonds appeared firstly to construct the side chains. Thereafter, the formations of the disulfide bonds and the carbon-carbon double bonds are both applied in the construction of the side chains for stabilizing the alpha conformation of the polypeptide.

In the process of constructing the covalent bonds of the side chains, the coupled side chains are usually on an identical plane of the alpha-helix, so the amino acids are usually coupled to the side chains at i/i+3, i/i+4, i/i+7 and i/i+11, wherein the positions of i/i+4 and i/i+7 are relatively common.

However, each stabilization method has certain limitations. For example, in the stabilization method of the amido bond, the amido bond is readily hydrolyzed and thus it is possible for the amido bond to fail to function in the human body because of being hydrolyzed; in the stabilization method having two cysteines as the formation base of the side chain, the side chain always has the aromatic group and thus generates a great impact on the property of the polypeptide; the triazole, formed by the side chain based on the reaction between azide and alkyne, is a pharmacophore in nature and generates a possibly greater impact on the druggability of the polypeptide.

As a result, none of the prior side chain technologies is universal so far, so that it is necessary to develop more polypeptide stabilization technologies for the researchers to choose according to different situations.

### SUMMARY OF THE PRESENT INVENTION

An object of the present invention is to provide a method for stabilizing an alpha helix of a polypeptide by constructing side chains containing chiral sulfoxide.

Another object of the present invention is to provide a polypeptide compound having a modification of side chains in a stable alpha helical structure and a preparation method thereof.

Accordingly, in order to accomplish the above objects, the present invention provides a method for stabilizing an alpha helix of a polypeptide comprising steps of:

(1) connecting an unnatural amino acid to an amino terminus of a polypeptide and end-capping via an acetylation;

(2) processing a product of the step (1) with a thiolene reaction and then obtaining a polypeptide compound having a modification of thioether side chains, wherein the thioether side chains are coupled with amino acids at i/i+4;

(3) oxidizing the polypeptide compound having the modification of the thioether side chains and then obtaining a polypeptide compound having a modification of R-configured sulfoxide side chains or S-configured sulfoxide side chains; and

(4) separating and purifying a product of the step (3) and then obtaining the polypeptide compound having the modification of the R-configured sulfoxide side chains.

Preferably, the unnatural amino acid of the step (1) has a structure of:

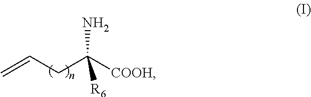

wherein R₆ is hydrogen or methylene; and n is a positive integer between 1˜6.

Preferably, the thiolene reaction of the step (2) comprises a photopolymerization reaction between the product of the step (1) and cysteines, or between the product of the step (1) and a cysteine derivative, and then a synthesis of the polypeptide compound having the modification of the thioether side chains by forming amido bonds.

Preferably, reaction equations of the step (2) and the step (3) are showed as follows.

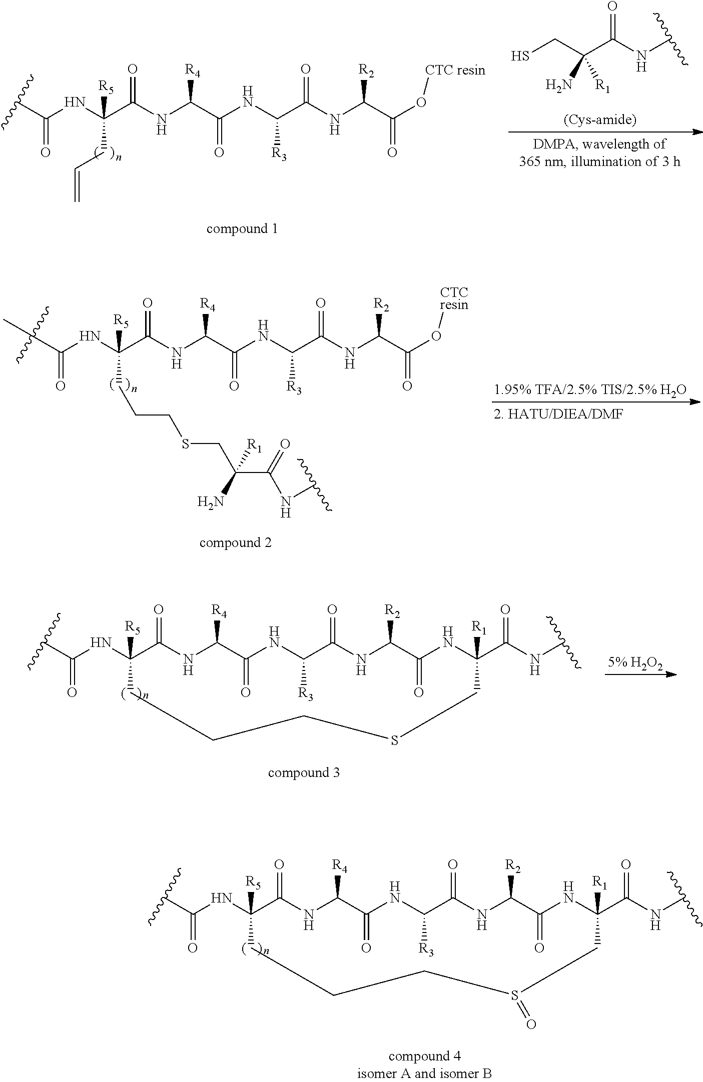

The present invention further provides a polypeptide compound having a modification of side chains, wherein the polypeptide compound has a structure of:

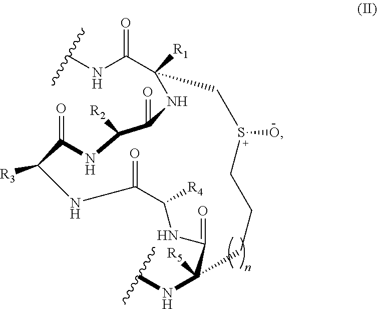

wherein R₁ and R₅ are independently hydrogen or methyl; R₂˜R₄ are independently amino acid residues; n is a positive integer between 1˜6; and the sulfoxide is R-configured.

Preferably, the polypeptide has a length of no more than 20 amino acids.

Preferably, n is 3 or 4.

The present invention further provides a preparation method of the polypeptide compound comprising steps of:

(i) connecting an unnatural amino acid to an amino terminus of the polypeptide and end-capping via an acetylation, wherein the unnatural amino acid has a structure of:

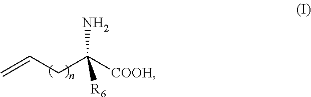

wherein R₆ is hydrogen or methylene; and n is a positive integer between 1˜6;

(ii) processing a product of the step (i) with a thiolene reaction and then obtaining a polypeptide compound having a modification of thioether side chains, wherein the thioether side chains are coupled with amino acids at i/i+4;

(iii) oxidizing the polypeptide compound having the modification of the thioether side chains and then obtaining a polypeptide compound having a modification of R-configured sulfoxide side chains or S-configured sulfoxide side chains; and

(iv) separating and purifying a product of the step (iii) and then obtaining the polypeptide compound having the modification of the R-configured sulfoxide side chains.

Preferably, the thiolene reaction of the step (ii) comprises a photopolymerization reaction between the product of the step (i) and cysteines, or between the product of the step (i) and a cysteine derivative, and then a synthesis of the polypeptide compound having the modification of the thioether side chains by forming amido bonds.

Preferably, reaction equations of the step (ii) and the step (iii) are showed as follows.

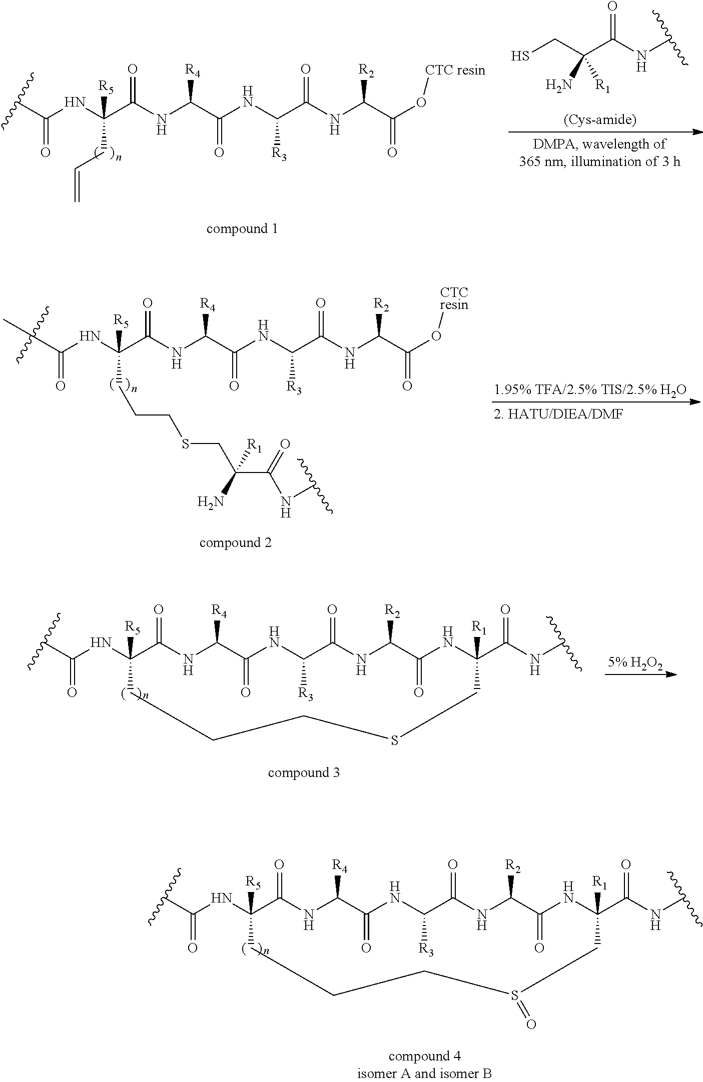

These and other objectives, features, and advantages of the present invention will become apparent from the following detailed description, the accompanying drawings, and the appended claims.

### BRIEF DESCRIPTION OF THE DRAWINGS

FIG. 1 is a circular dichroism (CD) diagram of Ac-cyclo(1,5)-monoS₅AAAC-NH₂ sulfoxide A and B in a phosphate buffered saline (PBS, ph=7.4, 10 mM) when n=3 according to an Example 1 of the present invention.

FIG. 2 is a CD diagram of the Ac-cyclo(1,5)-monoS₅AAAC-NH₂ sulfoxide B in the PBS and a 50% trifluoroethanol (TFE) solution according to the Example 1 of the present invention.

FIG. 3 is a CD diagram of the Ac-cyclo(1,5)-monoS₅AAAC-NH₂ sulfoxide B (ASOB), Ac-cyclo(1,5)-monoS₅AGAC-NH₂ sulfoxide B (GSOB) and Ac-cyclo(1,5)-monoS₅AIAC-NH₂ sulfoxide B (ISOB) according to the Example 1 of the present invention.

FIG. 4 is a CD diagram of Ac-cyclo(1,5)-monoS₆AAAC-NH₂ sulfoxide A and B in the PBS (10 mM) when n=4 according to an Example 2 of the present invention.

FIG. 5 is a high performance liquid chromatography (HPLC) diagram of purified Ac-cyclo(1,5)-monoS₅AAAC-NH₂ according to the Example 1 of the present invention.

FIG. 6 is a liquid chromatography-mass spectrum (LC-MS) diagram of the polypeptide Ac-cyclo(1,5)-monoS₅AAAC-NH₂ according to the Example 1 of the present invention.

FIG. 7 is an LC-MS diagram of the polypeptide Ac-cyclo(1,5)-monoS₅AAAC-NH₂ sulfoxide A according to the Example 1 of the present invention.

FIG. 8 is an LC-MS diagram of the polypeptide Ac-cyclo(1,5)-monoS₅AAAC-NH₂ sulfoxide B according to the Example 1 of the present invention.

FIG. 9 is an LC-MS diagram of the polypeptide Ac-cyclo(1,5)-monoS₅AAAC-NH₂ sulfoxide A according to the Example 2 of the present invention.

FIG. 10 is an LC-MS diagram of the polypeptide Ac-cyclo(1,5)-monoS₅AAAC-NH₂ sulfoxide B according to the Example 2 of the present invention.

FIG. 11 is an HPLC diagram of the polypeptides Ac-cyclo(1,5)-monoS₅AAAC-NH₂ sulfoxide A and B according to the Example 1 of the present invention.

### DETAILED DESCRIPTION OF THE PREFERRED EMBODIMENT

According to prior researches, polypeptide molecules, different from small molecular drugs, have large contact areas and low toxicity, but the polypeptides always fail to have a secondary structure or a tertiary structure of proteins. Besides, in a physiological environment, a short polypeptide shorter than twenty amino acids always fails to have a stable conformation. Thus, how to obtain the polypeptides which are shorter than twenty amino acids and have stable conformations in an aqueous phase, maintains to be a hot issue of polypeptide chemical research. As one of most important secondary structures of the proteins, an alpha helix plays an important role in many essential physiological processes, such as signal transmission and protein-protein mutually action. Thus the physiological processes would become better controlled when the polypeptides have the stable alpha helixes, wherein stabilizing the polypeptide alpha helix by constructing side chains of covalent bonds is one of most frequently applied techniques.

According to the present invention, a pentapeptide is taken as a model, and a stability of an alpha helix of the pentapeptide is significantly improved by constructing side chains of the present invention, wherein the side chains have a high tolerance to sequence. A method for stabilizing the polypeptide, provided by the present invention, has a good prospect application. Firstly, a polypeptide compound having stable thioether side chains is obtained via a thiolene reaction; further the thioether is oxidized into a sulfoxide having chirality. Then it is proved that the side chains of the chiral sulfoxide is well capable of stabilizing the alpha helix of the polypeptide and has the high tolerance to the polypeptide sequence via a circular dichroism (CD).

A following general formula of structure shows positions of the side chains on the polypeptide.

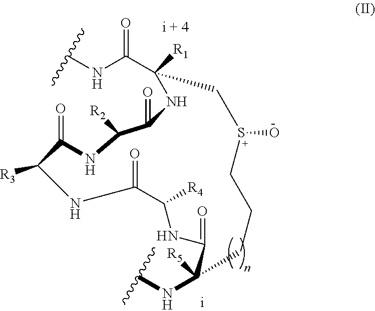

In the above general formula, R1 and R5 are independently hydrogen or methyl; R2˜R4 are independently residues of twenty natural amino acids; and n is a positive integer between 1˜6. Preferably, n is 3 or 4, and the sulfoxide is R-configured.

A preparation method of the polypeptide compound having the stable side chains comprises following key steps.

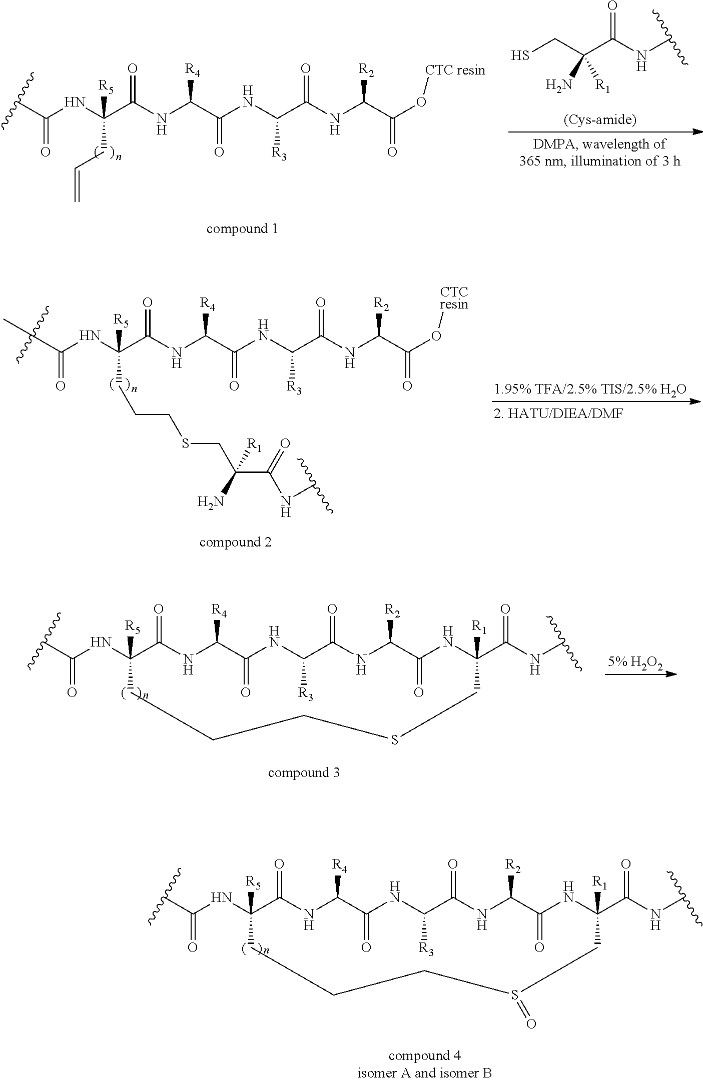

The above compound 1 is obtained by connecting an unnatural amino acid to an amino terminus of the polypeptide and then end-capping via an acetylation, wherein the natural amino acids are accomplished via connecting peptides to a solid-phase protected by fluorenylmethyloxycarbonyl (Fmoc) group; the unnatural amino acid (monoSₙ₊₂) has a structure of:

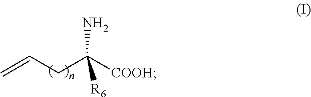

the unnatural amino acid is connected to the resin via connecting the peptides to the solid-phase, wherein R₆ is hydrogen or methylene; and

n is a positive integer between 1˜6, preferably 3˜4.

The compound 2 is obtained by reacting the compound 1 with cysteine (Cys-amide) and derivatives thereof. Reaction conditions thereof are set as: to 0.5 mmol of the compound 1 adding 1.5 mmol of a photo-initiator of 2,2-dimethoxy-2-phenylacetophenone (DMPA), 1.5˜2.5 mmol of the Cys-amide derivatives, then a solvent of 10 ml of N,N-dimethylformamide (DMF); degassing and then reacting for 3 h in 365 nm ultra-violet (UV) light; and filtering off the reaction liquid to obtain the compound 2.

To a 10 ml shear fluid of trifluoroacetic acid/triisopropylsilane/H₂O (TFA/TIS/H₂O) in a volume ratio of 9.5:0.25:0.25 is added the above obtained compound 2. Blow the shear liquid dry with nitrogen and precipitate with 10 ml of ether/n-hexane in a volume ratio of 4:1; extract the precipitation thoroughly and then dissolve into 200 ml of DMF to obtain a mixture; degas, add 1.0 g of 2-(7-Aza-1H-benzotriazole-1-yl)-1,1,3,3-tetramethyluronium hexafluorophosphate (HATU) and 0.9 ml of diisopropylethylamine (DIEA) in a cold bath to the mixture and then slowly increase a temperature of the mixture to a room temperature to react for 36 h; when an LCMS detection indicates that the reaction is completed, spin the solvent dry, purify via HPLC, dry by freezing and then obtain the pure compound 3, i.e., the polypeptide compound having a modification of the thioether side chains.

The compound 3 dissolved into 4 ml of H₂O is added into 1 ml of 30% hydrogen peroxide to react for 3 h. The compound 4, i.e., the polypeptide compound having a modification of the sulfoxide side chains, is obtained after purifying via HPLC. The compound 4 comprises two isomers, wherein a first peak indicates the compound 4A and a second peak indicates the compound 4B.

The obtained compound 4 is dissolved into of PBS (PH=7.4, 10 mM) for obtaining a CD diagram. The compound 4B has a good alpha helix; a sequence generated by the method of the present invention has a higher content of helixes than the sequence generated by the conventional stabilization method via the conventional amido bond.

The CD result proves that the side chains of the chiral sulfoxide are well capable of stabilizing the alpha helix of the polypeptide and have the high tolerance to the polypeptide sequence.

As a conclusion, the present invention provides the method for stabilizing the polypeptide into the alpha helix by constructing the side chains. Within a certain range, the method is able to control a content of the alpha helix by adjusting a chain length. Compared to the conventional stabilizing method via the conventional amido bond, the method of the present invention generates better stabilization; different from the prior arts, the method of the present invention has exclusive benefits comprising the simple side chains, the high content of the alpha helix, the high sequence tolerance and the capability of controlling the content of the alpha helix within the certain range by adjusting the chain length.

Combined with the examples, the present invention is further illustrated as follows. One skilled in the art will understand that the examples of the present invention as shown in the drawings and described above is exemplary only and not intended to be limiting. In the following examples, the experimental methods without notes of specific conditions are usually executed under the common conditions or the conditions suggested by manufacturer.

Unless being specifically defined, the professional and scientific terms of the invention are already known by the ones skilled in the art. Besides, its examples have been shown and described for the purposes of illustrating the functional and structural principles of the present invention and are subject to change without departure from such principles. Therefore, this invention includes all modifications encompassed within the spirit and scope of the following claims.

### Example 1

According to the Example 1 of the present invention, a polypeptide of Ac-cyclo(1,5)-monoS₅AAAC-NH₂ sulfoxide stabilized by side chains of chiral sulfoxide is synthesized when n=3, wherein the monoS₅ has a structure of:

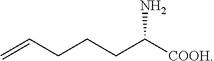

A process for synthesizing the Ac-cyclo(1,5)-monoS₅AAAC-NH₂ sulfoxide comprises steps of:

firstly, synthesizing Ac-monoS₅AAA-CTC resin via a solid-phase peptide synthesis, comprising steps of:

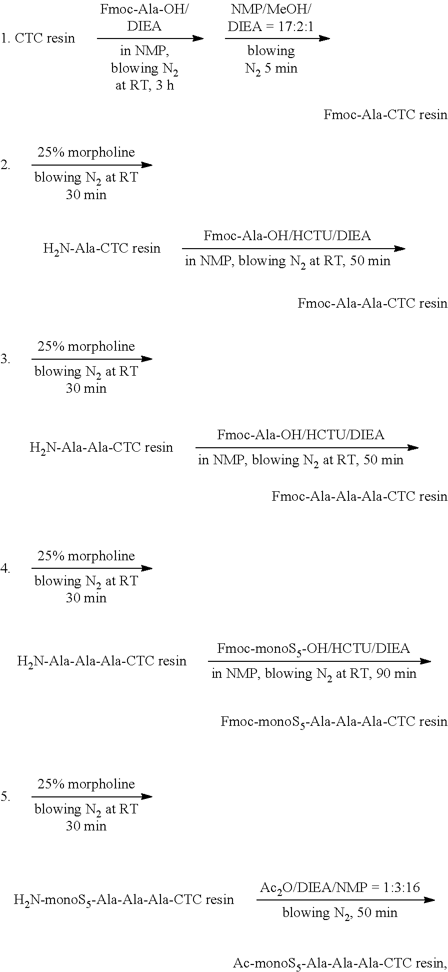

wherein the step (1) for connecting a first amino acid comprises adding 1.0 g of CTC resin to a 100 ml peptide connecting tube, adding 20 ml of N-methylpyrrolidone (NMP), blowing N₂ until swelling for 30 min; filtering off solvent, then adding a solution of 9331 mg of Fmoc-Ala-OH, 9.6 ml of NMP and 2.11 ml DIEA, blowing N₂ for 3.0 h; washing; suctioning the solvent inside the peptide connecting tube dry and washing the resin three times with NMP (10 ml*3), one minute per time; sealing; after the washing is completed, filtering off the reaction liquid, then adding 10 ml of NMP/MeOH/DIEA in a volume ratio of 17/2/1, blowing N₂ for 5 min; filtering off the reaction liquid and washing, for follow-up reactions;

the step (2) for connecting a second amino acid comprises adding an NMP solution containing 25% (volume percentage) morpholine, blowing N₂ therein for 30 min and washing, for accomplishing de-protection; uniformly mixing Fmoc-Ala-OH (0.4M in NMP) solution, O-(6-Chloro-1-hydrocibenzotriazol-1-yl)-1,1,3,3-tetramethyluronium hexafluorophosphate (HCTU) (0.38M in NMP), and DIEA in a volume ratio of 7.5 ml/7.5 ml/1 ml, adding the mixture into the resin and blowing N₂ therein for 50 min, for accomplishing a litigation; filtering off the reaction liquid and washing, for follow-up reactions;

the step (3) for connecting a third amino acid comprises identical steps with the step (2);

the step (4) for connecting an unnatural amino acid monoS₅ comprises, after de-protection, uniformly mixing Fmoc-monoS₅—OH (0.4M in NMP) solution, HCTU (0.38M in NMP) and DIEA in a volume ratio of 5.0 ml/5.0 ml/0.71 ml, adding the mixture into the resin and blowing N₂ therein for 120 min; filtering off the reaction liquid, for follow-up reactions;

the step (5) for end-capping via an acetylation comprises, after de-protection, washing and filtering off NMP; adding 10 ml of a mixture of Ac2O/DIEA/NMP (1:3:16) and blowing N₂ therein for 50 min; and

the step (6) comprises filtering off the reaction liquid, washing the resin successively with NMP (10 ml), dichloromethane (DCM) (10 ml) and methanol (MeOH) (10 ml), suctioning dry and storing, for follow-up reactions;

secondly, slicing off 30 mg of the resin and adding the sliced resin to a 1.5 ml eppendorf tube; adding 0.5 ml of TFA/TIPS/H₂O (9.5:0.25:0.25) into the tube for an oscillation reaction for 0.5 h; precipitating; removing the resin by filtering, blowing the shear liquid dry with N₂ and then adding 1 ml of a cold mixture of ether/n-hexane (4:1); centrifuging to remove a supernatant, precipitating and suctioning solids dry;

wherein the solids are dissolved into 0.5 ml of deuterated dimethyl sulfoxide (DMSO) for an nuclear magnetic detection:

¹H NMR (500 MHz, DMSO) δ 8.07 (d, J=7.3 Hz, 1H), 8.02 (d, J=7.4 Hz, 1H), 7.97 (d, J=7.9 Hz, 1H), 7.81 (d, J=7.6 Hz, 1H), 5.80-5.71 (m, 1H), 5.02-4.89 (m, 2H), 4.23-4.15 (m, 4H), 1.82 (s, 3H), 1.59 (d, J=9.4 Hz, 1H), 1.45 (d, J=5.4 Hz, 1H), 1.39-1.28 (m, 2H), 1.28-1.13 (m, 11H);

thirdly, constructing side chains via a thiol-ene reaction, comprising:

(i) processing the solids with photopolymerization according to an equation of:

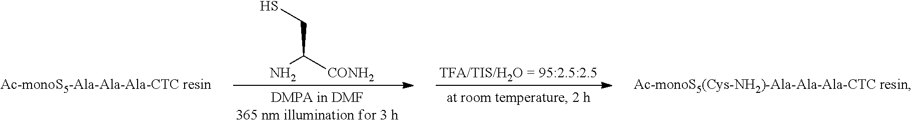

and (ii) obtaining a reaction intermediate of:

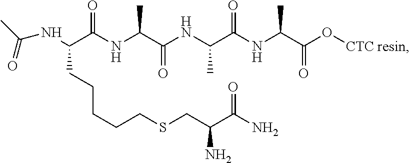

specifically comprising steps of: to a 25 ml flask adding 1.0 g of Ac-monoS₅AAA-CTC resin and successively adding 780 mg of Cys-amide, 380 mg of DMPA and 50 ml of DMF; blowing N₂ therein three times to remove oxygen within the solvent; providing the flask at 20 cm under a high-pressure mercury lamp and stirring the flask to react for 3 h; transferring the reacted resin into a peptide connecting tube, filtering off the reaction liquid, washing alternatively with 10 ml of DMF, 10 ml of DCM and 10 ml of MeOH and then suctioning dry, so as to obtain Ac-monoS₅(Cys-NH₂)AAA-CTC resin; wherein

30 mg of Ac-monoS₅(Cys-NH₂)AAA-CTC resin are sliced, precipitated, and processed with a nuclear magnetic detection, wherein double-bond peaks at two positions of 5.80-5.71 (m, 1H) and 5.02-4.89 (m, 2H) on ¹H spectrum are observed to detect whether the reaction is completed; the thiol-ene reactions are repeated when the double-bond peaks still exist; the rest resin with 10 ml of shear liquid for 3 h are reacted when the double-bond peaks disappear and the reaction is completed, as showed in following ¹H spectrum; the shear liquid is blown dry with N₂, added 10 ml of a cold mixture of ether/n-hexane in a volume ratio of 4:1 to precipitate, and centrifuged to remove a supernatant; and finally solids are suctioned dry for storage;

¹H NMR (400 MHz, DMSO) δ 8.14 (s, 3H), 8.08 (d, J=7.3 Hz, 1H), 8.05-7.96 (m, 2H), 7.93-7.85 (m, 2H), 7.67 (s, 1H), 4.30-4.11 (m, 5H), 2.93-2.91 (m, 1H), 2.87-2.77 (m, 1H), 1.84 (s, 3H), 1.65-1.54 (m, 1H), 1.54-1.39 (m, 3H), 1.39-1.16 (m, 13H);

and (iii) synthesizing a cyclopeptide of Ac-cyclo(1,5)-monoS₅AAAC-NH₂ via forming amido bonds which specifically comprises steps of:

transferring the solids obtained via the above thiol-ene reactions into a 1000 ml flask, adding 500 ml of DMF, blowing N₂ therein three times; under a protection of N₂, adding 1.0 g of peptide connecting reagent of HATU in a cold bath; stirring for 10 min and then adding 0.9 ml of DIEA; and slowly increasing a temperature of the mixture to a room temperature and then reacting for 12 h; wherein

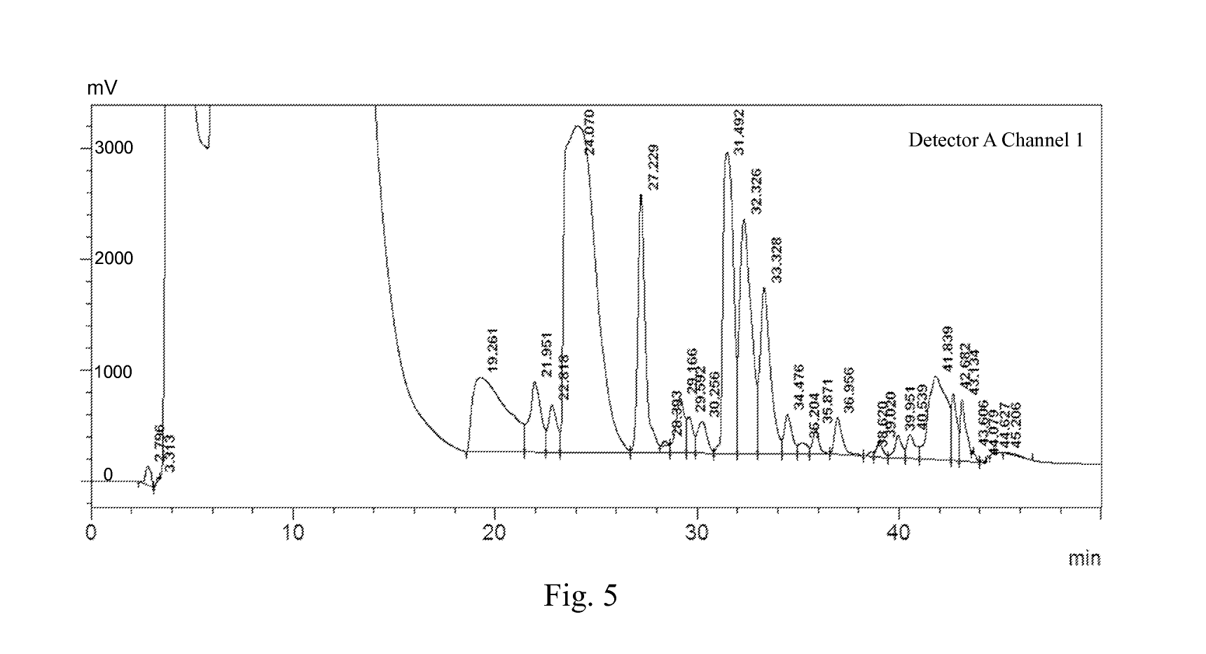

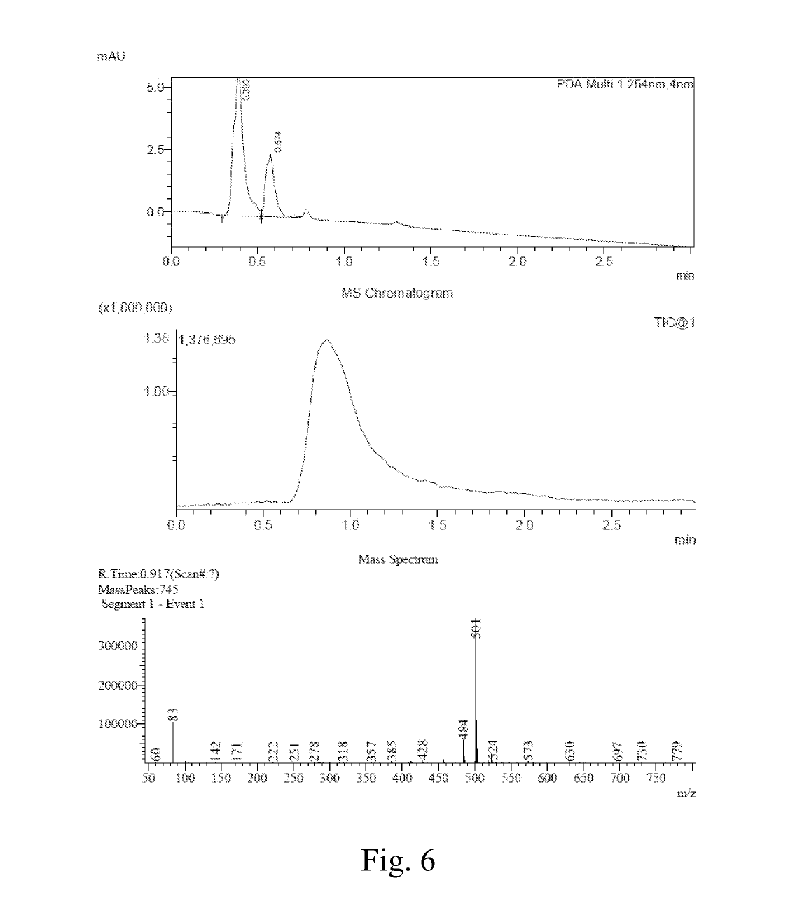

the reaction liquid is spun dry and then purified via HPLC, 250*10 mm C18 reversed phase chromatogram, solution A: 0.1% TFA H₂O, solution B: 0.1% TFA acetonitrile; solvent gradient: 0-45 min 5-50%; Rt=31.492, wherein FIG. 5 shows an HPLC chromatogram of the cyclopeptide Ac-cyclo(1,5)-monoS₅AAAC-NH₂ and FIG. 6 shows detection results of an LCMS detection thereof;

Table 1 showing an analysis result of FIG. 5 is listed as follows;

TABLE 1
Analysis Result of HPLC of Ac-cyclo(1,5)-monoS₅AAAC-NH₂

| peak name | reserved time | height | start | end | area % |
|---|---|---|---|---|---|
| P | 31.492 | 2712473 | 30.808 | 31.983 | 10.7948 |

and the product is dried via freezing; then 2 mg of the dried product is dissolved in 0.5 ml of deuterated dimethyl sulfoxide (DMSO) for nuclear magnetic detection:

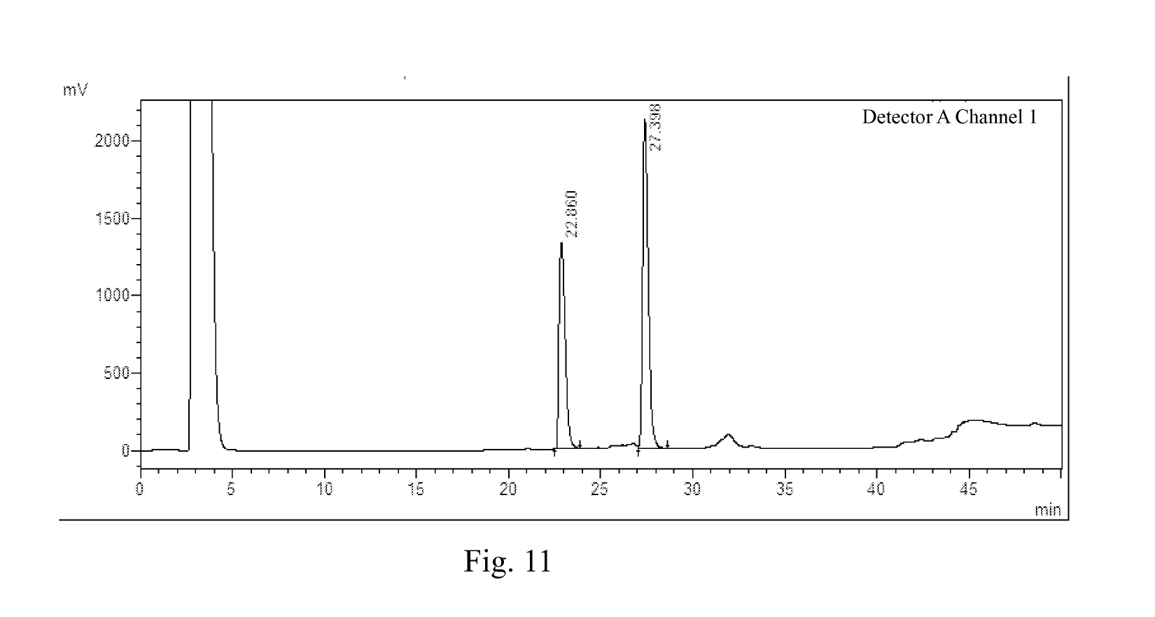

¹H NMR (400 MHz, DMSO) δ 8.38 (d, J=7.3 Hz, 1H), 8.20 (d, J=5.9 Hz, 1H), 8.00 (d, J=7.8 Hz, 1H), 7.95 (d, J=8.1 Hz, 1H), 7.39 (d, J=6.4 Hz, 1H), 7.21 (s, 1H), 7.08 (s, 1H), 4.29-4.05 (m, 5H), 2.84 (dd, J=13.9, 4.4 Hz, 1H), 2.76-2.65 (m, 1H), 1.82 (s, 3H), 1.74-1.69 (m, 1H), 1.62-1.12 (m, 18H); and fourthly, dissolving 3 mg of the cyclopeptide of Ac-cyclo(1,5)-monoS₅AAAC-NH₂ into 4 ml of H₂O and adding 1 ml of 30% H₂O₂ to react for 3 h; purifying via HPLC and then obtaining two diastereoisomers of cyclopeptide having side chains containing chiral sulfoxide, Ac-cyclo(1,5)-monoS₅AAAC-NH₂ sulfoxide A and Ac-cyclo(1,5)-monoS₅AAAC-NH₂ sulfoxide B, as showed in FIG. 11.

Table 2 showing an analysis result of FIG. 11 is listed as follows.

TABLE 2
Analysis Result of HPLC of Ac-cyclo(1,5)-monoS₅AAAC-NH₂
sulfoxide A and B

| peak name | reserved time | height | start | end | area % |
|---|---|---|---|---|---|
| A | 22.860 | 31087340 | 22.483 | 23.850 | 41.0151 |
| B | 27.398 | 44707504 | 27.050 | 28.600 | 58.9849 |

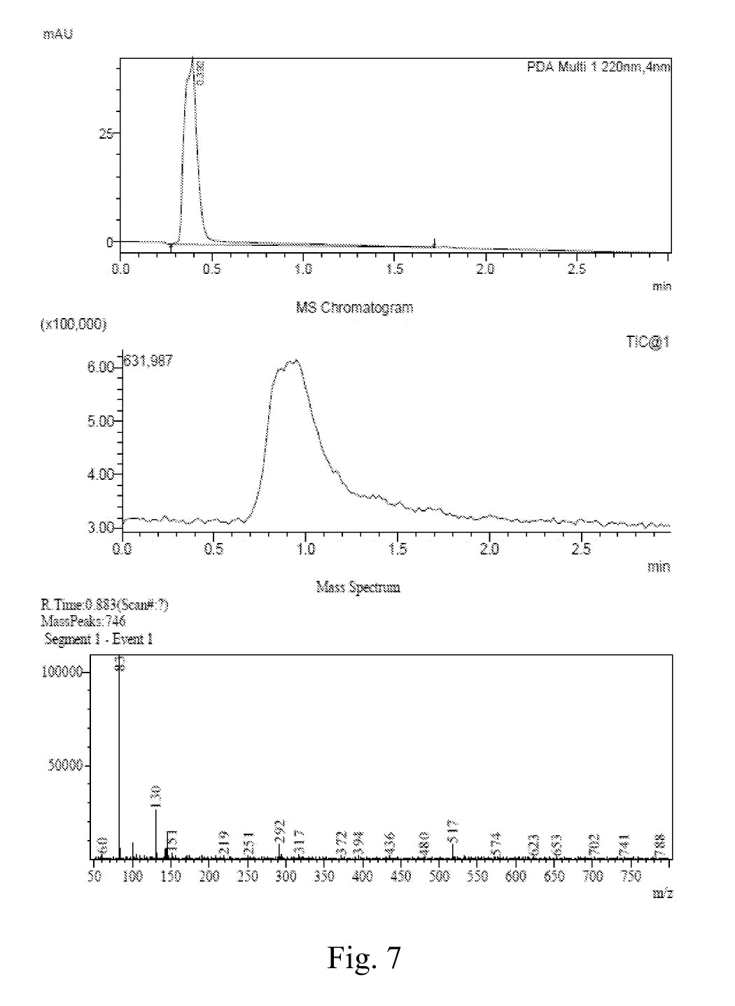

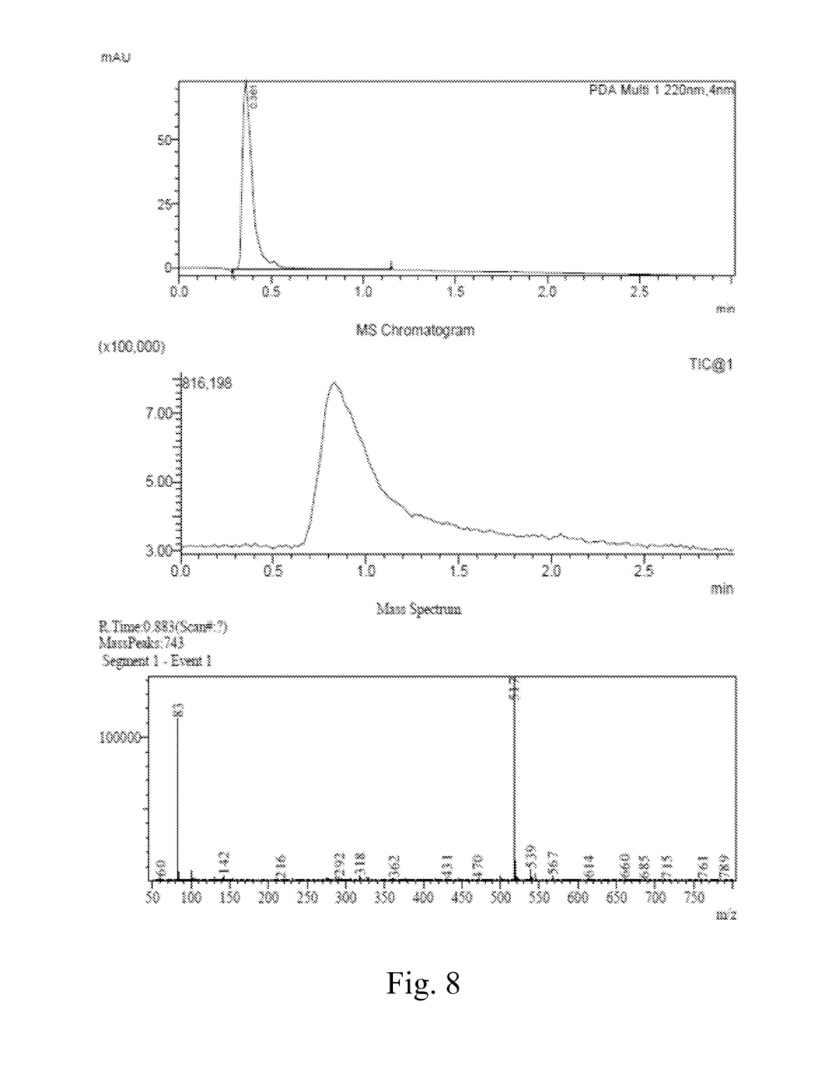

FIG. 7 shows an LC-MS diagram of the polypeptide Ac-cyclo(1,5)-monoS₅AAAC-NH₂ sulfoxide A and FIG. 8 shows an LC-MS diagram of the polypeptide Ac-cyclo(1,5)-monoS₅AAAC-NH₂ sulfoxide B. ¹H NMR results thereof are showed as follows.

### Ac-cyclo(1,5)-monoS₅AAAC-NH₂ sulfoxide A

¹H NMR (500 MHz, DMSO) δ 8.36 (d, J=7.5 Hz, 1H), 8.22 (d, J=7.5 Hz, 2H), 7.95 (d, J=8 Hz, 1H), 7.53 (d, J=5 Hz, 1H), 7.18 (d, J=7.5 Hz, 2H), 4.51-4.39 (m, 1H), 4.25-4.19 (m, 2H), 4.01-3.95 (m, 1H), 3.15-3.03 (m, 2H), 2.87-2.81 (m, 1H), 2.70-2.64 (m, 1H), 1.81 (s, 3H), 1.75-1.62 (m, 2H), 1.45-1.34 (m, 5H), 1.32 (s, 3H), 1.25-1.21 (m, 9H).

### Ac-cyclo(1,5)-monoS₅AAAC-NH₂ sulfoxide B

¹H NMR (500 MHz, DMSO) δ 8.42 (d, J=6.9 Hz, 1H), 8.12 (d, J=6.2 Hz, 1H), 8.07 (d, J=5.7 Hz, 1H), 7.95 (d, J=8.7 Hz, 1H), 7.28 (d, J=10.4 Hz, 2H), 7.20 (s, 1H), 4.57 (t, J=8.4 Hz, 1H), 4.13 (dd, J=14.5, 7.4 Hz, 3H), 4.06-3.97 (m, 1H), 3.08-2.94 (m, 2H), 2.87 (dd, J=13.2, 7.1 Hz, 1H), 2.70-2.60 (m, 1H), 1.87 (s, 3H), 1.79 (dd, J=18.3, 11.6 Hz, 2H), 1.60-1.47 (m, 3H), 1.32 (s, 3H), 1.24 (t, J=6.5 Hz, 9H).

Two isomers, ASOA/ASOB, of the Ac-cyclo(1,5)-monoS₅AAAC-NH₂ sulfoxide are dissolved in PBS (10 mM) for a CD detection, wherein 0.1-0.5 mg of samples of the two isomers are respectively dissolved in 0.5 ml of 10 mmol PBS for the CD detection at the wavelength of 190 nm-250 nm and then contents of alpha helixes are counted according to following formulae, as showed in Table 3.

f_{H}=([θ]_{obs215}-[θ]_{C})/([θ]_{∞215}-[θ]_{C});

[θ]_{obs215}=θ/(10*C*Nₚ*ι);

[θ]_{∞215}=(−44000+250T)(1−κ/Nₚ);

[θ]_{C}=2220−53T=1054;

wherein T=22° C.; κ=4.0; Nₚ=6 (for pentapeptide); ι=0.1 (cm); C is a molarity M of the samples of the two isomers.

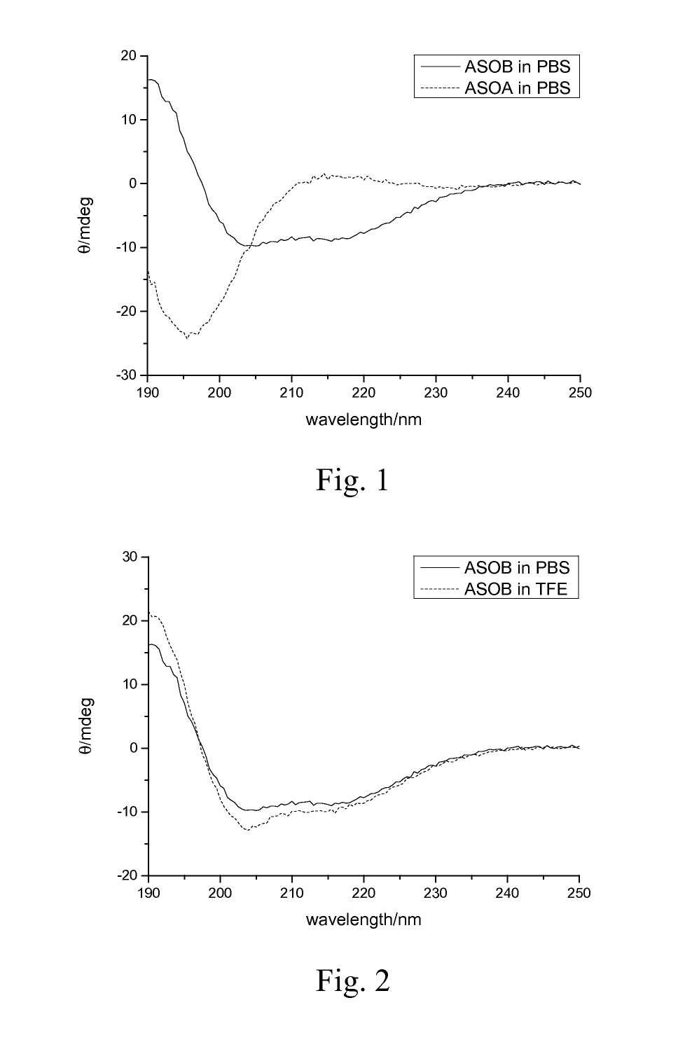

FIG. 1 indicates that only the isomer B whose peak emerges later in the HPLC separation has stable alpha helixes.

By comparing the CD diagrams of the Ac-cyclo(1,5)-monoS₅AAAC-NH₂ sulfoxide B in PBS and a 50% TEF buffer solution, it is proved that the TFE buffer solution brings no improvements to the alpha helix, which means that the stabilizing method via the side chains has increased the alpha helix of the short peptides to a best level.

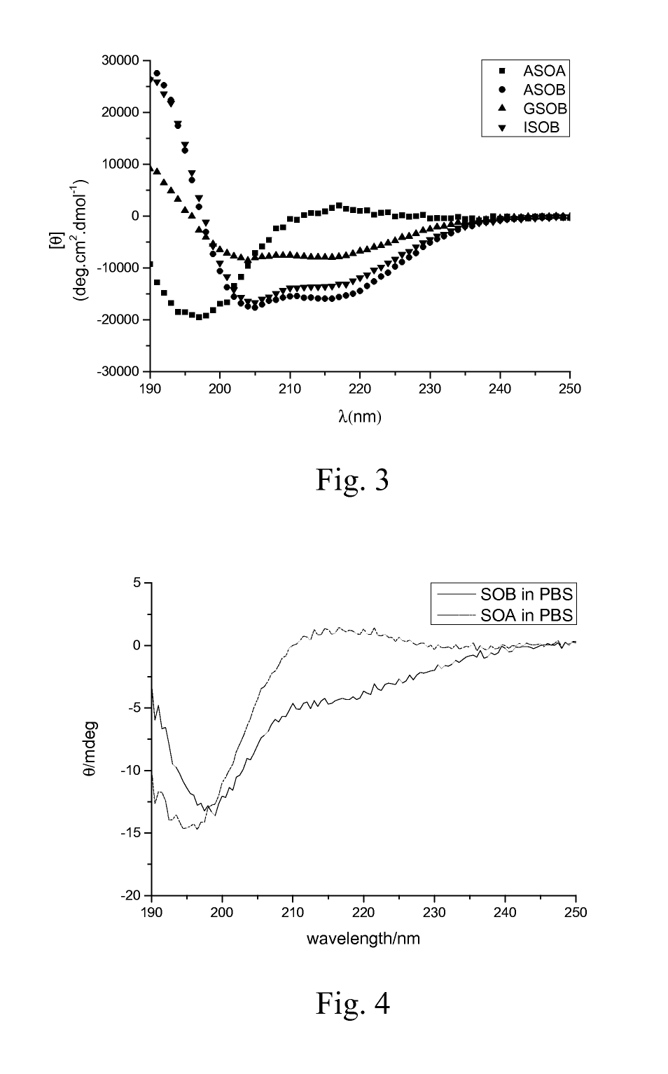

Changing an intermediate amino acid of the pentapeptide is against forming Ac-cyclo(1,5)-monoS₅AGAC-NH₂ sulfoxide B (GSOB) and Ac-cyclo(1,5)-monoS₅AIAC-NH₂ sulfoxide B (ISOB) via the secondary structures G and I of the alpha helix, but the GSOB and the ISOB according to the Example 1 of the present invention have identically high contents of the alpha helixes, as showed in Table 3 and FIG. 3.

By comparing the method provided by the present invention to the conventional stabilizing method via the amido bond and observing ASOA/control in Table 3, in the condition of identical intermediate sequences, the stabilizing method of the present invention improves the effects of stabilizing the alpha helix 14% compared to the conventional stabilizing method via the amido bond.

TABLE 3
Relative Content of Alpha Helixes of Polypeptides in PBS
(pH = 7.4, 22° C., 10 mM)

| | | | | | relative content |
|---|---|---|---|---|---|
| polypeptide | [θ]₂₁₅ | [θ]₂₀₇ | [θ]₁₉₀ | [θ]₂₁₅/[θ]₂₀₇ | of alpha helix* |
| ASOA | 672 | −4667 | −14594 | −0.14 | −0.04 |
| ASOB | −15666 | −16054 | 25987 | 0.98 | 1.00 |
| GSOB | −7920 | −7777 | 9121 | 1.02 | 0.51 |
| ISOB | −13558 | −15556 | 26306 | 0.87 | 0.87 |
| control | −13537 | −13684 | 39352 | 0.99 | 0.86** |

*ASOB is the standard and [θ]₂₁₅(x)/[θ]₂₁₅(ASOB) is the relative content of alpha helix
**control represents the cyclopeptide of Ac-(cyclo2,6)-R[KAAAD]-NH₂ having the side chains of the amido bond

### Example 2

According to the Example 2 of the present invention, when n=4, a polypeptide stabilized by side chains of chiral sulfoxide is synthesized and detected via CD diagrams.

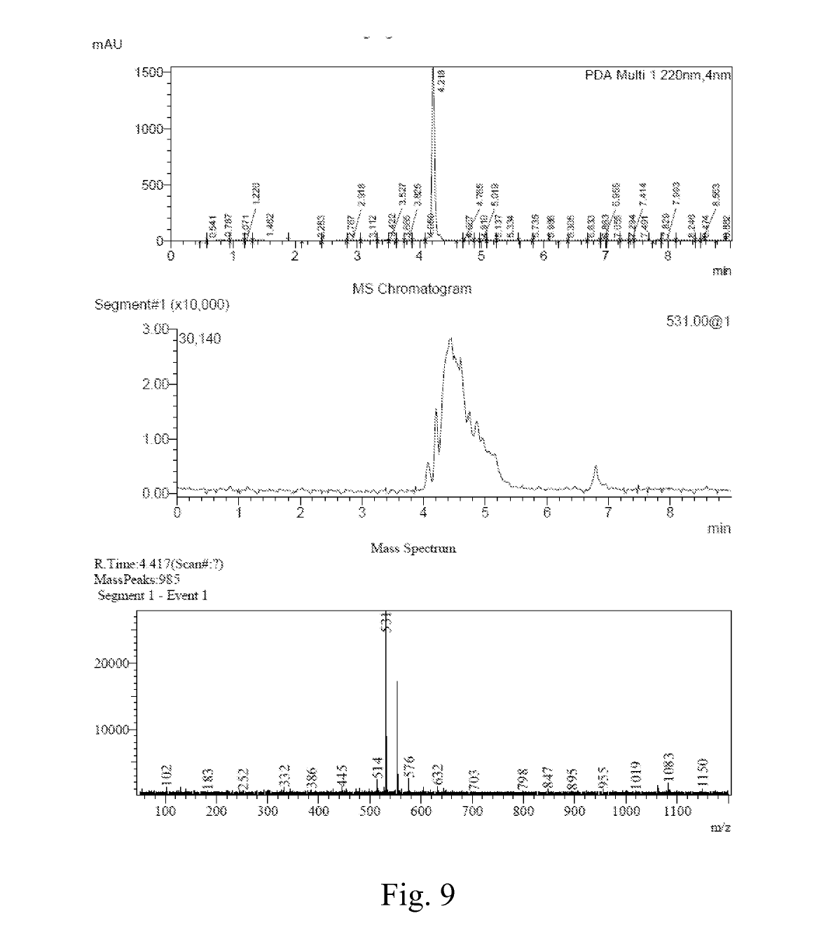

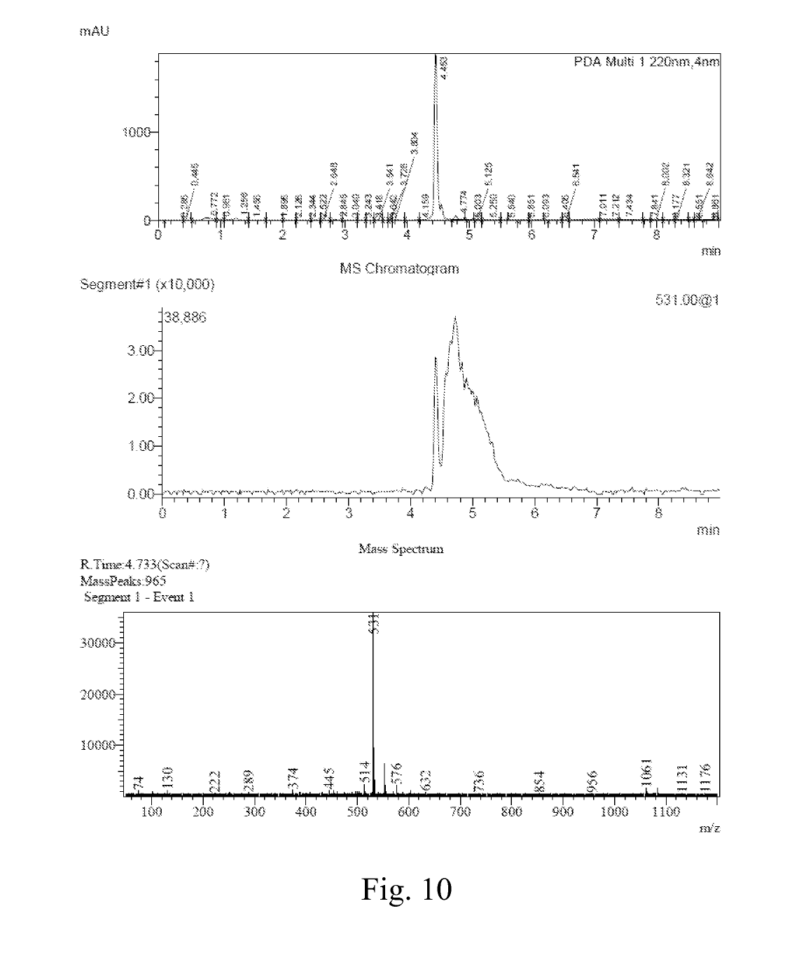

According to the Example 2 of the present invention, monoS₅ is replaced by monoS₆ having one more carbon atom; similarly to the Example 2, two diastereoisomers are obtained and named as Ac-cyclo(1,5)-monoS₆AAAC-NH₂ sulfoxide A and Ac-cyclo(1,5)-monoS₆AAAC-NH₂ sulfoxide B according to peak-emerging time. FIGS. 9 and 10 show results of LCMS detections thereof and ¹H NMR results thereof are showed as follows.

### Ac-cyclo(1,5)-monoS₆AAAC-NH₂ sulfoxide A

¹H NMR (500 MHz, DMSO) δ 8.42 (d, J=7.6 Hz, 1H), 8.33 (d, J=8.2 Hz, 1H), 8.19 (d, J=6.4 Hz, 1H), 7.99 (d, J=7.9 Hz, 1H), 7.24-7.16 (m, 3H), 4.68-4.61 (m, 1H), 4.30-4.08 (m, 4H), 3.17 (dd, J=13.8, 3.0 Hz, 1H), 3.02 (dd, J=13.9, 9.3 Hz, 1H), 2.71 (dd, J=13.7, 6.5 Hz, 2H), 1.81 (s, 3H), 1.76-1.67 (m, 1H), 1.67-1.52 (m, 2H), 1.52-1.40 (m, 3H), 1.40-1.18 (m, 13H).

### Ac-cyclo(1,5)-monoS₆AAAC-NH₂ sulfoxide B

¹H NMR (500 MHz, DMSO) δ 8.41 (d, J=7.4 Hz, 1H), 8.20 (d, J=6.8 Hz, 1H), 8.13 (d, J=8.7 Hz, 1H), 7.98 (d, J=7.7 Hz, 1H), 7.39 (s, 1H), 7.20 (d, J=7.5 Hz, 2H), 4.54 (t, J=9.7 Hz, 1H), 4.29-4.13 (m, 4H), 3.08-2.93 (m, 2H), 2.74 (td, J=13.3, 6.4 Hz, 2H), 1.82 (s, 3H), 1.64 (t, J=18.4 Hz, 3H), 1.44 (d, J=6.9 Hz, 3H), 1.38-1.17 (m, 13H).

According to FIG. 4, it is indicated that only the diastereoisomers B has effects of stabilizing alpha helix.

It will thus be seen that the objects of the present invention have been fully and effectively accomplished. Its examples have been shown and described for the purposes of illustrating the functional and structural principles of the present invention and is subject to change without departure from such principles. Therefore, this invention includes all modifications encompassed within the spirit and scope of the following claims.

What is claimed is:

1. A method for stabilizing a polypeptide into an alpha helix, comprising steps of:
(1) connecting an unnatural amino acid to an amino terminus of the polypeptide and end-capping via an acetylation;
(2) processing a product of the step (1) with a thiolene reaction, obtaining a polypeptide compound having a modification of thioether side chains, wherein the thioether side chains are coupled with amino acids at i/i+4;
(3) oxidizing the polypeptide compound having the modification of the thioether side chains and then obtaining a polypeptide compound having a modification of R-configured sulfoxide side chains or S-configured sulfoxide side chains; and
(4) separating and purifying a product of the step (3) and then obtaining the polypeptide compound having the modification of the R-configured sulfoxide side chains.

2. The method, as recited in claim 1, wherein the unnatural amino acid of the step (1) has a structure of: 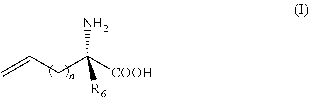
wherein R₆ is hydrogen or methylene; and n is a positive integer between 1˜6.

3. The method, as recited in claim 1, wherein the thiolene reaction of the step (2) comprises a photopolymerization reaction between the product of the step (1) and cysteine or between the product of the step (1) and a cysteine derivative and then a synthesis of the polypeptide compound having the modification of the thioether side chains by forming amido bonds.

4. The method, as recited in claim 1, wherein reaction equations of the steps (2) and (3) are:

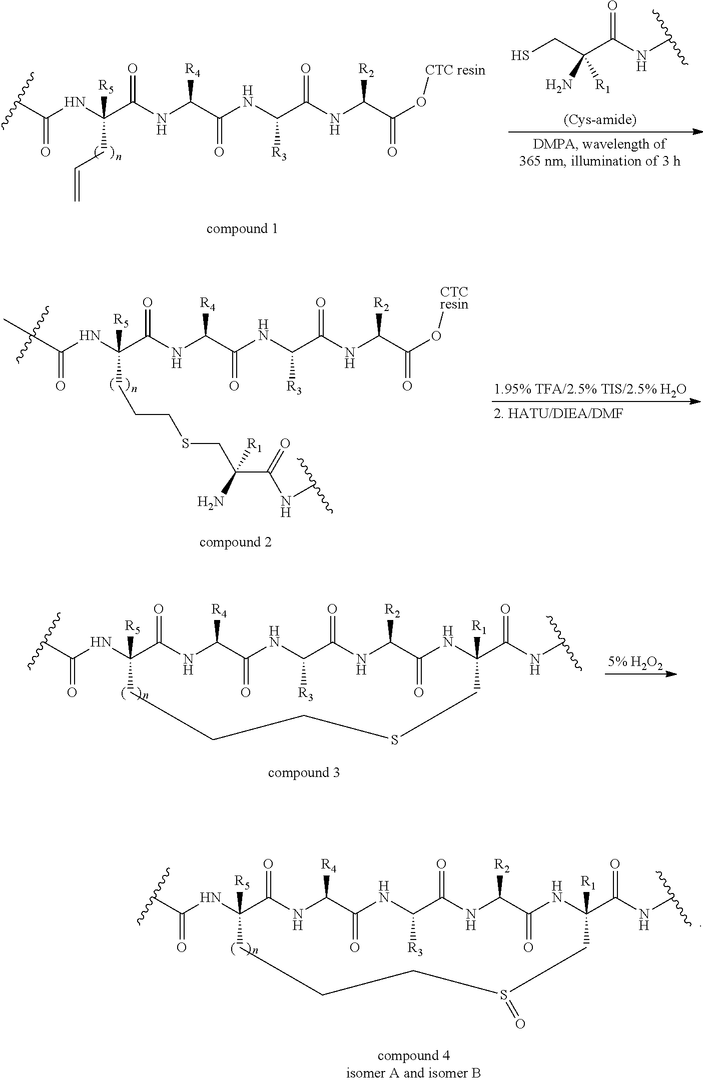

5. A polypeptide compound having a modification of side chains, wherein the polypeptide has a structure of: 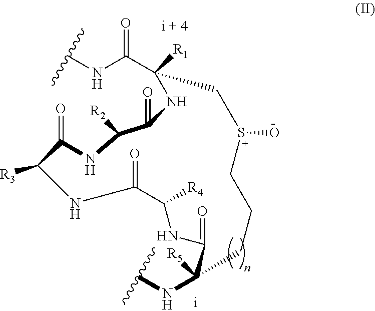
wherein R₁ and R₅ are independently and respectively hydrogen or methyl; R₂˜R₄ are independently and respectively residues of amino acids; n is a positive integer between 1˜6; and sulfoxide is R-configured.

6. The polypeptide compound, as recited in claim 5, wherein said polypeptide has a length of no more than 20 amino acids.

7. The polypeptide compound, as recited in claim 5, wherein n is 3 or 4.

8. A preparation method of the polypeptide compound as recited in claim 5, comprising steps of:
(i) connecting an unnatural amino acid to an amino terminus of the polypeptide and end-capping via an acetylation, wherein the unnatural amino acid has a structure of: 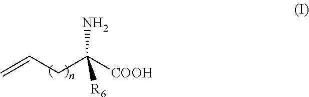
wherein R₆ is hydrogen or methylene and n is a positive integer between 1˜6;
(ii) processing a product of the step (i) with a thiolene reaction and obtaining a polypeptide compound having a modification of thioether side chains, wherein the side chains are coupled with amino acids at i/i+4;
(iii) oxidizing the polypeptide compound having the modification of the thioether side chains and obtaining a polypeptide compound having a modification of R-configured sulfoxide side chains or S-configured sulfoxide side chains; and
(iv) separating and purifying a product of the step (iii) and obtaining the polypeptide compound having the modification of the R-configured sulfoxide side chains.

9. The preparation method, as recited in claim 8, wherein the thiolene reaction of the step (ii) comprises a photopolymerization reaction between the product of the step (i) and cysteines, or a cysteine derivative, and then a synthesis of the polypeptide compound having the modification of the thioether side chains by forming amido bonds.

10. The preparation method, as recited in claim 8, wherein reaction equations of the steps (ii) and (iii) are: [chemical structure]
